(19) United States
(12) Patent Application Publication
(10) Pub. No.: US 2014/0039486 A1
(43) Pub. Date: Feb. 6, 2014

(54) ELECTROSURGICAL SYSTEM

(71) Applicant: Paul James Wootton, Newport (GB)

(72) Inventor: Paul James Wootton, Newport (GB)

(73) Assignee: GYRUS MEDICAL LIMITED, Cardiff (GB)

(21) Appl. No.: 14/110,159

(22) PCT Filed: Apr. 4, 2012
(86) PCT No.: PCT/GB2012/000313

§ 371 Date: Oct. 16, 2013

(30) Foreign Application Priority Data
Apr. 7, 2011 (GB) 1105877.3

(51) Int. Cl. A61B 18/18

(52) U.S. Cl. CPC A61B 18/18
USPC 606/33

(57) ABSTRACT

An electrosurgical instrument includes an active electrode and an instrument return electrode, while the resectoscope also includes a resectoscope return electrode. The generator includes a source of radio frequency energy capable of producing either a coagulating RF waveform or a cutting RF waveform, and first, second and third output connections connected to the active electrode, the instrument return electrode, and the resectoscope return electrode respectively. The generator also includes a switching device, and a controller, the controller being such that when a cutting RF waveform is selected, the switching device directs the cutting RF waveform between the first and second output connections and hence the active electrode and the instrument return electrode. When a coagulating RF waveform is selected, the switching device directs the coagulating RF waveform between the first and third output connections and hence the active electrode and the resectoscope return electrode.

### TECHNICAL FIELD

This invention relates to an electrosurgical system and in particular to a system for endoscopic urological surgery using a resectoscope.

### BACKGROUND TO THE INVENTION AND PRIOR ART

Systems for endoscopic urological surgery using a resectoscope are well known in the art, examples being given in U.S. Pat. Nos. 5,007,907 and 6,322,494. Such systems to include an electrosurgical instrument deployable by means of a resectoscope, and an electrosurgical generator powering the instrument. A generator suitable for powering a urological instrument is described in U.S. Pat. No. 7,211,081. Instruments used in electrosurgical urology surgery are either bipolar, in which case two electrodes are present at the distal end of the instrument, or monopolar, in which case one electrode is present on the instrument and a second electrode is provided in the form of a patient return plate.

### SUMMARY OF INVENTION

The present invention provides an improvement to these types of instrument, and includes an electrosurgical generator, an electrosurgical instrument coupled to the generator, and a resectoscope through which the electrosurgical instrument is presented, the electrosurgical instrument including an active electrode and an instrument return electrode, the resectoscope including a resectoscope return electrode, wherein the generator includes a source of radio frequency energy capable of producing either a coagulating RF waveform or a cutting RF waveform, and first, second and third output connections connected to the active electrode, the instrument return electrode, and the resectoscope return electrode respectively, the generator including a switching means, and a controller, the controller being such that when a cutting RF waveform is selected, the switching means directs the cutting RF waveform between the first and second output connections and hence the active electrode and the instrument return electrode, and when a coagulating RF waveform is selected, the switching means directs the coagulating RF waveform between the first and third output connections and hence the active electrode and the resectoscope return electrode.

Thus, the system employs the instrument return electrode when tissue cutting is required, and the resectoscope return electrode when tissue coagulation is required. The close proximity of the active electrode and instrument return electrode provides for maximum safety when tissue cutting is being performed. However, when tissue coagulation is desired, it is more advantageous for the electrodes to be further spaced, allowing for the coagulating current to travel further through the tissue to form a deeper tissue effect. In a convenient arrangement, the resectoscope is comprised of an electrically-conducting material, and the resectoscope return electrode is constituted by the body of the resectoscope. A resectoscope typically includes an elongate tube through which the instrument is deployed, and the tube can conveniently constitute the resectoscope return electrode.

The active electrode is conveniently in the form of a loop electrode, although other formations such as roller electrodes or button electrodes are also common. The generator and electrosurgical instrument are typically such that the instrument is designed to be operated in a conductive fluid, with the conductive fluid completing the current path between the electrodes.

The switching means is conveniently a relay system, located within the electrosurgical generator. However, as an alternative, the switching means can be constituted by a separate unit located between the generator and the instrument.

### DESCRIPTION OF THE DRAWINGS

Embodiments of the invention will now be further described, by way of example only, with reference to the accompanying drawings, in which:

FIG. 1 is a perspective view of an electrosurgical system in accordance with the present invention,

FIG. 2 is an exploded view of a resectoscopic instrument used as part of the electrosurgical system of FIG. 1,

FIG. 3 is a schematic circuit diagram of a generator used as part of the electrosurgical system of FIG. 1, and

FIG. 4 is a schematic diagram of a switching circuit forming part of the electrosurgical generator of FIG. 1.

### DESCRIPTION OF EMBODIMENTS

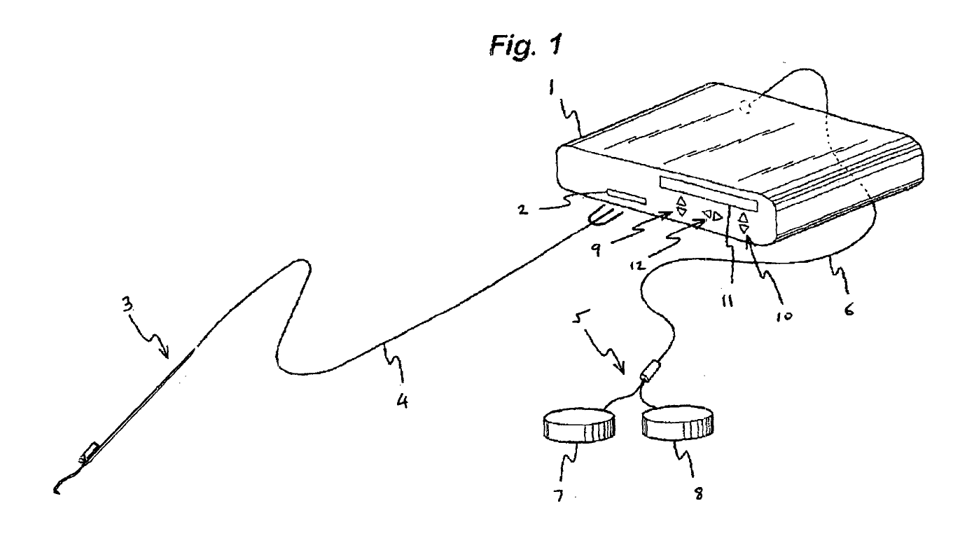

Referring to FIG. 1, a generator 1 has an output socket 2 providing a radio frequency (RF) output for an instrument 3 via a connection cord 4. Activation of the generator may be performed from the instrument 3 via a connection in cord 4 or by means of a footswitch unit 5, as shown, connected to the rear of the generator by a footswitch connection cord 6. In the illustrated embodiment footswitch unit 5 has two footswitch pedals 7 and 8 for selecting a coagulation mode and a cutting mode of the generator respectively. The generator front panel has push buttons 9 and 10 for respectively setting coagulation and cutting power levels, which are indicated in a display 11. Push buttons 12 are provided as a means for selection between alternative coagulation and cutting waveforms.

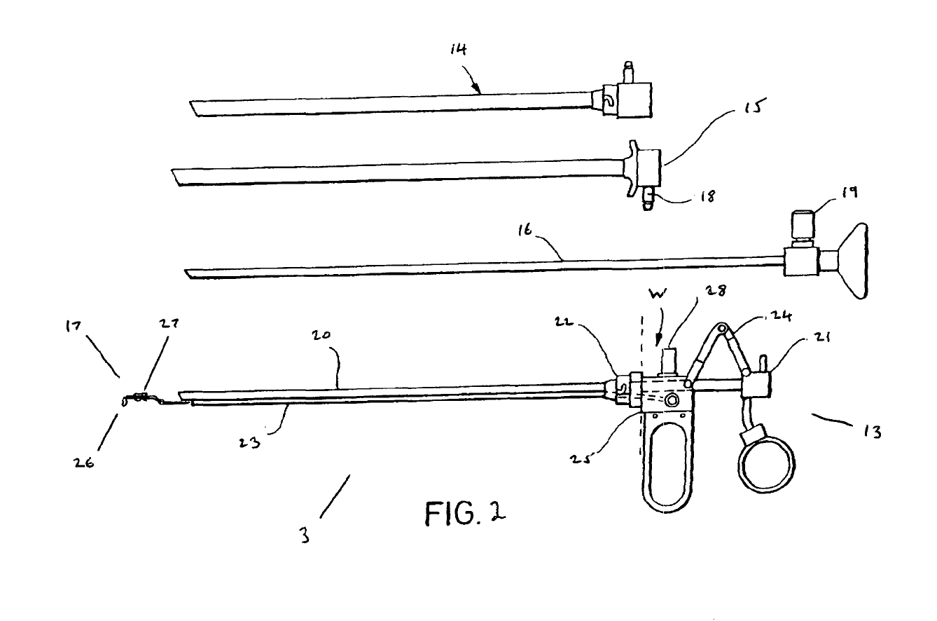

As shown in FIG. 2, the instrument 3 is deployed through a resectoscope 13 including an inner sheath 14, an outer sheath 15, and a rod lens telescope/light source assembly 16. The instrument 3 is part of a working element, indicated generally by the reference W, to the right of the dotted line shown in FIG. 1, and including a bipolar electrode assembly 17.

The sheaths 14 and 15 provide for the supply and aspiration of an operating site with a fluid medium via a connector 18. The outer sheath 15 locks over the inner sheath 14, forming a watertight seal. Typically, the inner sheath 14 has a diameter of 24Fr, and the outer sheath 15 has a diameter of 27 Fr. The telescope assembly 16 provides the means of illuminating and viewing the operative site via a light source (not shown) connected thereto by a connector 19. The viewing angle of the telescope is generally at 30° to its axis.

The working element W may be either passive or active, that is to say the cutting stroke of the electrode may be as the result of a spring bias or against the force of a spring bias. The telescope assembly 16 includes a telescope support tube 20 having a telescope connector 21 at its proximal end, and a sealing block 22 located part way along the support tube 20, the inner sheath 14 being connected to the sealing block. Both of these interfaces are watertight. An electrode support tube 23 is attached to the underside of the telescope support tube 20 on the distal side of the sealing block 22 for the majority of its length. Two spring-loaded links 24 and an insulation block 25, located between the sealing block 22 and the telescope connector 21, make up the mechanism. The active mechanism is arranged so that the spring-loaded links 24 assist the forward stroke, while, in the passive version the links aid the backward stroke. In general, the range of travel is about 25 mm.

The bipolar electrode assembly 17 includes an active electrode 26 in the form of a loop, and a return electrode 27 located on the shaft of the electrode assembly. The electrodes 26 & 27 are connected to the generator 1 via cord 4 connected via socket 28. The electrode support tube 23 is also formed of electrically conductive material, and constitutes a further return electrode, also connected to the generator I via cord 4.

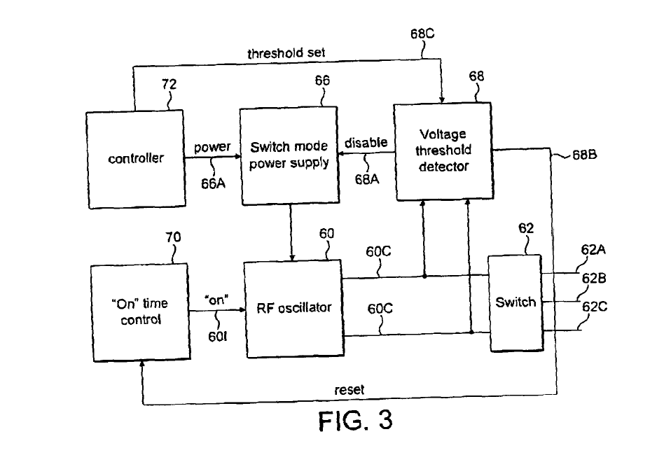

Referring to FIG. 3, the generator comprises a radio frequency (RF) output stage in the form of a power oscillator 60 having a pair of output lines 60C for coupling via switching circuit 62 to the instrument 3. Switching circuit 62 has three output connections 62A, 62B and 62C for connection to the electrodes of the instrument as will be described later. Power is supplied to the oscillator 60 by a switched mode power supply 66.

In the preferred embodiment, the RF oscillator 60 operates at about 400 kHz, with any frequency from 300 kHz upwards into the HF range being feasible. The switched mode power supply typically operates at a frequency in the range of from 25 to 50 kHz. Coupled across the output lines 60C is a voltage threshold detector 68 having a first output 68A coupled to the switched mode power supply 66 and a second output 68B coupled to an “on” time control circuit 70. A micro-processor controller 72 coupled to the operator controls and display (shown in FIG. 1) is connected to a control input 66A of the power supply 66 for adjusting the generator output power by supply voltage variation and to a threshold-set input 68C of the voltage threshold detector 68 for setting peak RF output voltage limits.

In operation, the microprocessor controller 72 causes power to be applied to the switched mode power supply 66 when electrosurgical power is demanded by the surgeon operating an activation switch arrangement which may be provided on a hand-piece or footswitch. A constant output voltage threshold is set independently of the supply voltage via input 68C according to control settings on the front panel of the generator. Typically, for desiccation or coagulation the threshold is set at a desiccation threshold value between 150 volts and 200 volts. When a cutting or vaporisation output is required the threshold is set to a value in the range of from 250 or 300 volts to 600 volts. These voltage values are peak values. Their being peak values means that for desiccation at least it is preferable to have an output RF waveform of low crest factor to give maximum power before the voltage is clamped at the values given. Typically a crest factor of 1.5 or less is achieved.

When the generator is first activated, the status of the control input 601 of the RF oscillator 60 (which is connected to the “on” time control circuit 70) is “on”, such that the power switching device which forms the oscillating element of the oscillator 60 is switched on for a maximum conduction period during each RF oscillation cycle. The power delivered to the tissue depends partly on the supply voltage applied to the RF oscillator 60 from the switched mode power supply 66 and partly on the tissue impedance. The voltage threshold for a desiccation output is set to cause trigger signals to be sent to the “on” time control circuit 70 and to the switched mode power supply 66 is when the voltage threshold is reached. The “on” time control circuit 70 has the effect of virtually instantaneously reducing the “on” time of the RF oscillator-switching device. Simultaneously, the switched mode power supply is disabled so that the voltage supplied to oscillator 60 begins to fall. The operation of the generator in this way is described in detail in our European Patent Application No. 0754437, the disclosure of which is hereby incorporated by way of reference.

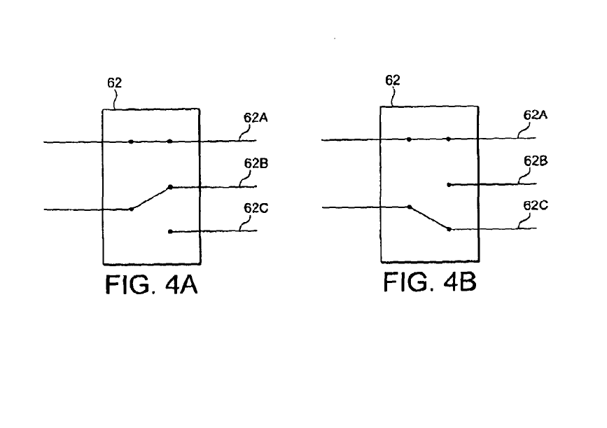

Output connections 62A and 62B from the generator 3 are electrically connected to the electrodes 26, & 27 respectively, via cord 4. Similarly, output connection 62C is electrically connected to the electrode support tube 23, also via cord 4. When it is desired to operate the instrument 3 in a cutting mode, footswitch 7 is depressed which causes a signal to be sent to the controller 72 which sets the switching circuit 62 its “cut” position. This is illustrated in FIG. 4A, in which the signals from the oscillator 60 are connected between output connections 62A and 62B. This means that the RF power signal is applied between the loop electrode 26 and the return electrode 27. Output connection 62C (and hence the electrode support tube 23) is not energised.

At the same time as the controller 72 sets the switching circuit to the position in FIG. 4A, it also sends a signal via line 68C to the voltage threshold detector 68 to set the peak output voltage limit to a relatively high “cutting” level. The control of this cutting signal is described in more detail in EP 0754437, referred to earlier. In cutting mode, the output from the generator is a relatively high voltage, with a consequent low current level, and the relatively small distance between the electrodes 26 & 27 ensures that the electrode assembly fires up and cuts tissue.

Alternatively, when it is desired to operate the instrument 3 in a coagulation mode, footswitch 8 is depressed which causes the controller 72 to set the switching circuit 62 to its “coag” state, as illustrated in FIG. 4B. In this set-up, the power signals from the oscillator are connected between output connections 62A and 62C. This means that the RF power signal is applied between the active electrode 26 and the electrode support tube 23. At the same time the controller sends a signal to the voltage threshold detector 68 to set the peak output voltage limit to a relatively lower “coagulating” level, again as more particularly described in EP 0754437. In “coag” mode, the output from the generator is a relatively lower voltage, with a corresponding is relatively higher current, and the relatively larger distance between the electrode 26 and the electrode support tube 23 ensures that an effective area of coagulation is produced.

1. An electrosurgical system comprising an electrosurgical generator, an electrosurgical instrument coupled to the generator, and a resectoscope through which the electrosurgical instrument is presented, the electrosurgical instrument including an active electrode and an instrument return electrode, the resectoscope including a resectoscope return electrode, wherein the generator includes a source of radio frequency energy capable of producing either a coagulating RF waveform or a cutting RF waveform, and first, second and third output connections connected to the active electrode, the instrument return electrode, and the resectoscope return electrode respectively, the generator including a switching means, and a controller, the controller being such that when a cutting RF waveform is selected, the switching means directs the cutting RF waveform between the first and second output connections and hence the active electrode and the instrument return electrode, and when a coagulating RF waveform is selected, the switching means directs the coagulating RF waveform between the first and third output connections and hence the active electrode and the resectoscope return electrode.

2. A system according to claim 1, wherein the resectoscope is comprised of an electrically-conducting material, and the resectoscope return electrode is constituted by the body of the resectoscope.

3. A system according to claim 1, wherein the active electrode is in the form of a loop electrode.

4. A system according to claim 1, wherein the generator and electrosurgical instrument are such that the instrument is designed to be operated in a conductive fluid, with the conductive fluid completing the current path between the electrodes.
